(19) United States
(12) Patent Application Publication
(10) Pub. No.: US 2021/0378207 A1
(43) Pub. Date: Dec. 9, 2021

(54) THERMAL SUPPORT FOR AN ANIMAL AND METHOD OF USE THEREOF

(71) Applicant: Modular Thermal Technologies, LLC, Pompano Beach, FL (US)

(72) Inventor: Colin M. Hall, Pompano Beach, FL (US)

(21) Appl. No.: 17/341,977

(22) Filed: Jun. 8, 2021

Related U.S. Application Data
(60) Provisional application No. 63/036,070, filed on Jun. 8, 2020.

(51) Int. Cl. A01K 1/02; A01K 1/00; F28C 3/00

(52) U.S. Cl. CPC A01K 1/0254; F28C 3/00; A01K 1/0076; A01K 1/029

(57) ABSTRACT

A thermal support for animals and method of use thereof is provided. The thermal support comprises a first portion, a strap portion, and a thermal insert. The first portion comprises a flexible fabric configured to conform to at least a portion of the animal. The first portion comprises an opening configured to receive the animal and a shape configured to support the animal when received by the opening. The first portion comprises a pocket configured to contact the animal when received by the opening. The strap portion is configured to enable lifting of the first portion. The thermal insert is configured to be heated and/or cooled. The thermal insert is removably positioned in the pocket of the first portion such that the thermal insert can transfer heat to or from the animal when received by the opening and when heated and/or cooled.

### CROSS-REFERENCE TO RELATED APPLICATIONS

This application claims priority to U.S. Provisional Patent Application No. 63/036,070, filed on Jun. 8, 2020, the contents of which are hereby incorporated by reference into this specification.

### FIELD OF USE

The present disclosure relates to a thermal support for an animal and a method of use thereof.

### BACKGROUND

Many owners like to carry their dogs while running errands and/or going for a walk. Additionally, some dogs require assistance while going for a walk. Carrying dogs and/or providing assistance to dogs to go for a walk presents challenges.

### SUMMARY

One aspect according to the present disclosure is directed to a thermal support for an animal. The thermal support comprises a first portion, a strap portion, and a thermal insert. The first portion comprises a flexible fabric configured to conform to at least a portion of the animal. The first portion comprises an opening configured to receive the animal and a shape configured to support the animal when received by the opening. The first portion comprises a pocket configured to contact the animal when received by the opening. The strap portion is attached to the first portion at two or more locations. The strap portion is configured to enable lifting of the first portion. The thermal insert is configured to be heated and/or cooled. The thermal insert is removably positioned in the pocket of the first portion such that the thermal insert can transfer heat or cold to or from the animal when received by the opening and when heated and/or cooled.

Another aspect according to the present disclosure is directed to a method for supporting an animal. The method comprises heating a thermal insert of a thermal support to a temperature greater than 30 degrees Celsius or cooling the thermal insert to a temperature less than 10 degrees Celsius. The thermal insert is placed into the pocket of a first portion of the thermal support. The animal is placed into an opening of the first portion. A strap portion of the thermal support is placed over the shoulder of a user and the animal is supported while the thermal insert transfers heat or cold to or from the animal.

It will be understood that the inventions disclosed and described in this specification are not limited to the aspects summarized in this Summary. The reader will appreciate the foregoing details, as well as others, upon considering the following detailed description of various non-limiting and non-exhaustive aspects according to this specification.

### BRIEF DESCRIPTION OF THE DRAWINGS

The features and advantages of the examples presented herein, and the manner of attaining them, will become more apparent, and the examples will be better understood, by reference to the following description taken in conjunction with the accompanying drawings, wherein:

FIG. 1 is perspective view illustrating an example of a thermal support for an animal according to the present disclosure;

FIG. 2 is a perspective view illustrating an example of the thermal support of FIG. 1 with an animal received by the thermal support;

FIG. 3 is perspective view illustrating an example of a thermal support for an animal according to the present disclosure;

FIG. 4 is a perspective view illustrating an example of the thermal support of FIG. 3 with an animal received by the thermal support; and

FIG. 5 is a partial cross-sectional view illustrating the thermal insert of FIG. 1.

Corresponding reference characters indicate corresponding parts throughout the several views. The exemplifications set out herein illustrate certain embodiments, in one form, and such exemplifications are not to be construed as limiting the scope of the appended claims in any manner.

### DESCRIPTION

Various examples are described and illustrated herein to provide an overall understanding of the structure, function, and use of the disclosed thermal supports and methods of using the thermal supports. The various examples described and illustrated herein are non-limiting and non-exhaustive. Thus, the invention is not limited by the description of the various non-limiting and non-exhaustive examples disclosed herein. Rather, the invention is defined solely by the claims. The features and characteristics illustrated and/or described in connection with various examples may be combined with the features and characteristics of other examples. Such modifications and variations are intended to be included within the scope of this specification. As such, the claims may be amended to recite any features or characteristics expressly or inherently described in, or otherwise expressly or inherently supported by, this specification. Further, Applicant reserves the right to amend the claims to affirmatively disclaim features or characteristics that may be present in the prior art. The various examples disclosed and described in this specification can comprise, consist of, or consist essentially of the features and characteristics as variously described herein.

Any references herein to “various examples”, “some examples”, “one example”, “an example”, “non-limiting examples”, or like phrases mean that a particular feature, structure, or characteristic described in connection with the example is included in at least one embodiment. Thus, appearances of the phrases “in various examples”, “in some examples”, “in one example”, “in an example”, “in a non-limiting example”, or like phrases in the specification do not necessarily refer to the same example. Furthermore, the particular described features, structures, or characteristics may be combined in any suitable manner in an example or examples. Thus, the particular features, structures, or characteristics illustrated or described in connection with one example may be combined, in whole or in part, with the features, structures, or characteristics of another example or other examples without limitation. Such modifications and variations are intended to be included within the scope of the present examples.

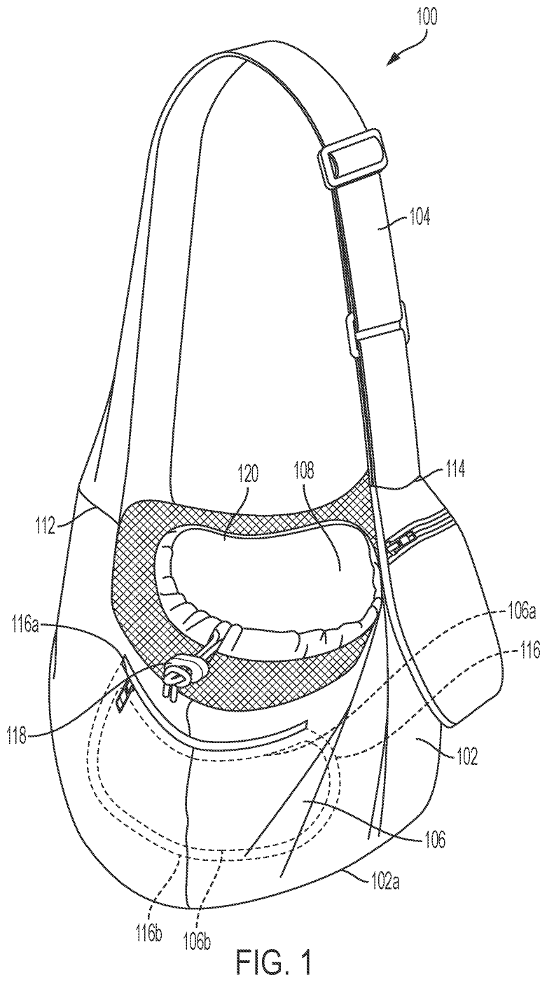

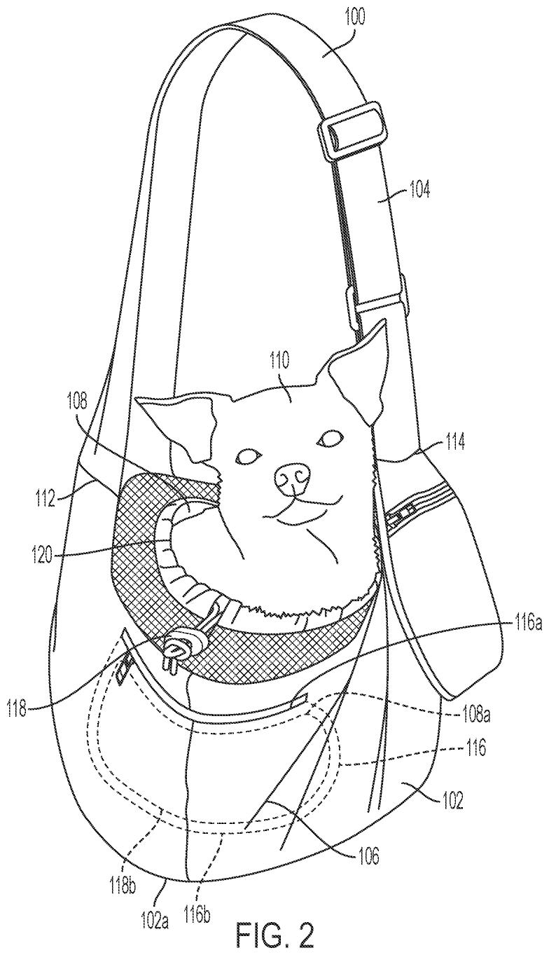

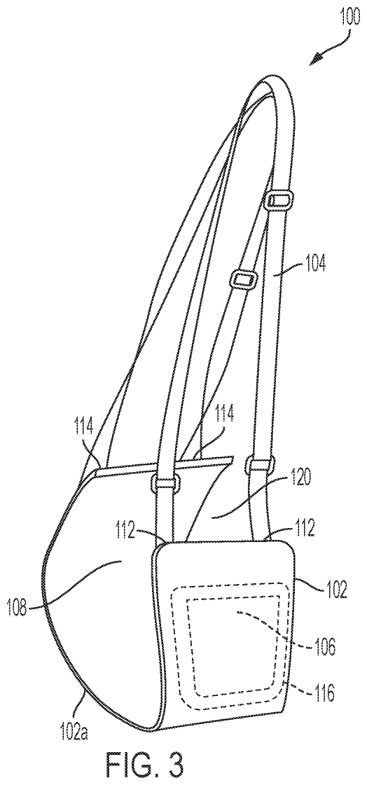

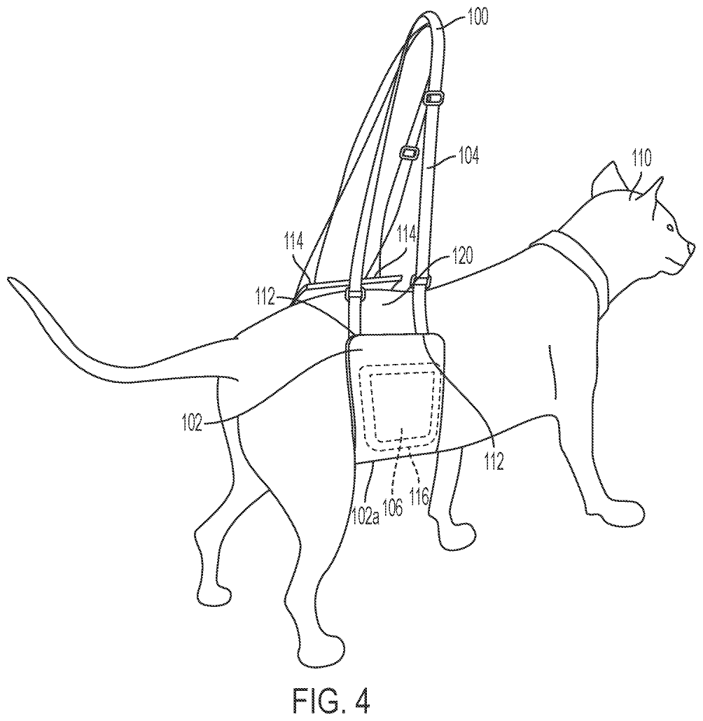

Supporting an animal (e.g., a pet), such as, for example, a dog, a rabbit, a ferret, a pig, a gerbil, a turtle, a lizard, a cat, or other animal, outdoors can expose the animal to undesirable temperatures such that the animal and/or person carrying the animal is uncomfortable (e.g., too hot, too cold). Additionally, an animal may experience trouble moving on their own (e.g., have an injury or medical condition), and the animal may need assistance to move. While moving the animal, it may be desirable to provide therapy to the animal to assist with the movement. Thus, the present disclosure provides a thermal support that can cool or heat an animal received in the thermal support and/or provide therapeutic cooling or heating to the animal received in the thermal support. In certain examples, the thermal support can be a thermal sling, such as, for example, as illustrated in FIGS. 1-2, where the temperature of the animal can be maintained at a desirable level by cooling or heating of the animal. In various examples, the thermal support can be a thermal hoist, such as, for example, as illustrated in FIGS. 3-4, where the animal can be supported while moving and receive therapeutic cooling or heat. The thermal support can enable a user to help an animal navigate stairs, get into a car, generally walk, or navigate other difficult terrain. For example, the thermal support can help arthritic pets walk.

Referring to FIGS. 1 and 3, a thermal support 100 for an animal is provided. As illustrated in FIG. 1, the thermal support 100 is a thermal sling. As illustrated in FIG. 3, the thermal support 100 is a thermal hoist. The thermal support 100 comprises a first portion 102, a strap portion 104, and a thermal insert 106. The thermal support 100 can be sized based on the desired application.

The first portion 102 comprises a flexible fabric configured to conform to at least a portion of the animal. For example, the flexible fabric can comprise cotton, cellulosic fibers, wool, silk, leather, bast fibers, synthetic fibers, other fabrics, or combinations thereof. The flexible fabric can enable the first portion 102 to conform to the body of an animal. For example, the first portion 102 can conform to the body of a dog 110 as illustrated in FIGS. 2 and 4. In certain examples, the flexible fabric is lightweight such that the thermal support 100 has a minimal weight burden to a user.

Referring back to FIGS. 1 and 3, the first portion 102 comprises an opening 120 configured to receive the animal and a shape configured to support the animal when received by the opening 120. For example, as illustrated in FIG. 1, the first portion 102 can be cup-shaped such that the animal can be received by the opening 120 and positioned in a cavity 108 within the first portion 102. A first region 102a of the first portion 102 can support the weight of the animal.

In various examples, as illustrated in FIG. 3, the first portion 102 can be U-shaped such that the animal can be received by the opening 120 and supported by the first region 102a of the first portion 102. The first region 102a can be configured to extend between the rear and front legs of a four-legged animal as illustrated in FIG. 4.

Referring yet again to FIGS. 1 and 3, the thermal support 100 comprises a strap portion 104 attached to the first portion 102 at two or more locations, such as, for example, location 112 and location 114. The locations 112 and 114 can be proximal to the opening 120 such that when the thermal support 100 is held by a user, the opening 120 is oriented upwards. The strap portion 104 can be attached to the first portion 102 by a fastener, sewing, other method, or combination thereof. The strap portion 110 is configured to enable lifting of the first portion 102. For example, the strap portion 110 can be placed on the shoulder of a user (e.g., person who owns the animal) to support the weight of the animal received in the first portion 102. For example, the user can carry the animal in the thermal support 100 in a hands free manner. In various examples, the strap portion 104 is adjustable (e.g., the length is adjustable).

The first portion 102 comprises a pocket 116 configured to contact the animal when received by the opening 120 and receive the thermal insert 106. The thermal insert 106 can be removably positioned in the pocket 116. The thermal insert 106 is configured to be heated or cooled. For example, the thermal insert 106 can comprise a thermal gel or other substance configured to store thermal energy and thereafter transfer heat to or from the animal. The thermal insert 106 can be heated (e.g., in a microwave) and/or cooled (e.g., in a freezer) separately from the first portion 102 and the strap portion 104. Thereafter, the thermal insert 106 can be inserted into the pocket 116 such that the thermal insert 106 can transfer heat or cold to or from an animal when received by the opening 120 and/or when the animal is in contact with the pocket 116. The thermal insert 106 can be reusable such that the thermal insert 106 can be reheated and/or recooled and reinserted into the pocket 116 as desired.

In various examples, the thermal gel can comprise water, a polymer (e.g., acrylic acid and acrylamide copolymer), an additional carrier (e.g., glycerin), and optionally additives (e.g., sodium alginate, gelatin, an antiseptic).

The thermal insert 106 is configured to conform to the at least a portion of the animal. For example, the thermal insert 106 can comprise a material that is flexible when heated and/or cooled. In various examples, the thermal insert 106 is flexible when cooled to a temperature of less than or equal to 0 degrees Celsius. Alternatively or in addition, the thermal insert 106 can have a shape suitable to conform to the body of the animal. In certain examples, the thermal insert 106 comprises a first concave side 106a and a first convex side 106b. In various examples, the pocket 116 comprises a second concave size 116a and a second convex side 116b, wherein the second concave size 116a is intermediate the opening 120 and the second convex side 116b. The second concave side 116a can comprise a shape corresponding to the first concave side 106a and the second convex side 116b can comprise a shape corresponding to the first convex side 106b. In various examples, the thermal insert 106 can be proximal to the first region 102a and form a floor for the animal to rest on.

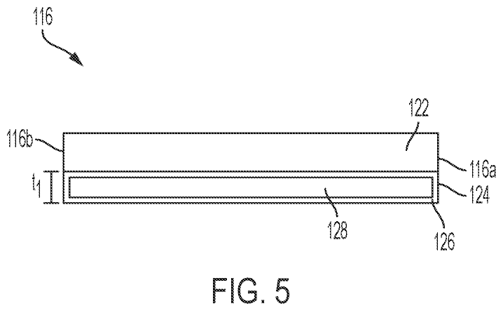

The thermal insert 106 can comprise multiple layers. For example, as illustrated in FIG. 5, the thermal insert 106 can comprise a thermal gel portion 124 and a support layer 122. The thermal gel portion 124 can be releasably detachable from the support layer 122, such as, for example, by a hook and loop fastener. The thermal gel portion 124 can have a thickness, t₁, in a range of 5 mm to 15 mm, such as, for example, 10 mm. The thermal gel portion 124 can comprise an enclosure 126 at least partially surrounding a thermal gel 128 positioned therein. The support layer 122 can support the shape of the thermal gel portion 124, which may be more flexible than the support layer 122.

In various examples, referring to FIGS. 1 and 3, the first portion 102 comprises an adjustable band 118 configured to control a size of the opening 120. For example, the first portion 102 can comprise a draw string. In certain examples, the thermal support 100 can comprise zippers pockets suitable to receive personal and pet accessories.

A method for supporting an animal, such as, for example, carrying or hoisting, is provided. The method comprises heating the thermal insert 106 to a temperature greater than 30 degrees Celsius or cooling the thermal insert 106 to a temperature of less than 10 degrees Celsius. The heating and/or cooling can be performed while the thermal insert 106 is outside of the pocket 116 and/or the first portion 102.

The heated and/or cooled thermal insert 106 can be placed into the pocket 116 of the first portion 102. The animal can be placed into the opening 120 of the first portion 102. The thermal insert 106 can transfer heat to or from the animal.

The strap portion 110 can be placed over the shoulder of a user and the animal in the first portion 102 can be supported in the thermal support 100. Thus, the animal can be supported by the user while the thermal insert 106 transfers heat to or from the animal.

In this specification, unless otherwise indicated, all numerical parameters are to be understood as being prefaced and modified in all instances by the term “about,” in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Also, any numerical range recited herein includes all sub-ranges subsumed within the recited range. For example, a range of “1 to 10” includes all sub-ranges between (and including) the recited minimum value of 1 and the recited maximum value of 10, that is, having a minimum value equal to or greater than 1 and a maximum value equal to or less than 10. Any maximum numerical limitation recited in this specification is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited. All such ranges are inherently described in this specification.

The grammatical articles “a”, “an”, and “the”, as used herein, are intended to include “at least one” or “one or more”, unless otherwise indicated, even if “at least one” or “one or more” is expressly used in certain instances. Thus, the foregoing grammatical articles are used herein to refer to one or more than one (i.e., to “at least one”) of the particular identified elements. Further, the use of a singular noun includes the plural, and the use of a plural noun includes the singular, unless the context of the usage requires otherwise.

As used herein, “intermediate” means that the referenced element is disposed between two elements but is not necessarily in contact with those elements. Accordingly, unless stated otherwise herein, an element that is “intermediate” a first element and a second element may or may not be adjacent to or in contact with the first and/or second elements, and other elements may be disposed between the intermediate element and the first and/or second elements.

One skilled in the art will recognize that the herein described thermal support, structures, operations/actions, and objects, and the discussion accompanying them, are used as examples for the sake of conceptual clarity and that various configuration modifications are contemplated. Consequently, as used herein, the specific examples/embodiments set forth and the accompanying discussion are intended to be representative of their more general classes. In general, use of any specific exemplar is intended to be representative of its class, and the non-inclusion of specific components, devices, apparatus, operations/actions, and objects should not be taken as limiting. While the present disclosure provides descriptions of various specific aspects for the purpose of illustrating various aspects of the present disclosure and/or its potential applications, it is understood that variations and modifications will occur to those skilled in the art. Accordingly, the invention or inventions described herein should be understood to be at least as broad as they are claimed and not as more narrowly defined by particular illustrative aspects provided herein.

What is claimed is:

1. A thermal support for an animal, the thermal sling comprising:
a first portion comprising a flexible fabric configured to conform to at least a portion of the animal, the first portion comprising an opening configured to receive the animal and a shape configured to support the animal when received by the opening, and the first portion comprising a pocket configured to contact the animal when received by the opening;
a strap portion attached to the first portion at two or more locations, the strap portion configured to enable lifting of the first portion; and
a thermal insert configured to be heated and/or cooled, the thermal insert removably positioned in the pocket of the first portion such that the thermal insert can transfer heat or cold to or from the animal when received by the opening and when heated and/or cooled.

2. The thermal support of claim 1, wherein the thermal insert is configured to be heated in a microwave and/or cooled in a freezer.

3. The thermal support of claim 1, wherein the first portion comprises an adjustable band configured to control a size of the opening.

4. The thermal support of claim 1, wherein the thermal insert is configured to conform to the at least a portion of the animal.

5. The thermal support of claim 1, wherein the thermal insert comprises a first concave side and a first convex side.

6. The thermal support of claim 1, wherein the pocket comprises a second concave size and a second convex side, wherein the second concave size is intermediate the opening and the second convex side.

7. The thermal support of claim 1, wherein the thermal insert is flexible when cooled to a temperature of less than or equal to 0 degrees Celsius.

8. The thermal support of claim 1, wherein the thermal insert comprises a thermal gel portion and a support layer, wherein the thermal gel portion is releasably detachable from the support layer.

9. The thermal support of claim 8, wherein the thermal gel portion is releasably detachable from the support layer by a hook and loop fastener.

10. The thermal support of claim 8, wherein the thermal gel portion comprises an enclosure comprising a flexible fabric and a thermal gel positioned within the enclosure.

11. The thermal support of claim 1, wherein the thermal support is a thermal sling and the first portion is cup shaped.

12. The thermal support of claim 1, wherein the thermal support is a thermal hoist and the first portion is a U-shaped.

13. The thermal support of claim 1, wherein the strap portion is attached proximal to the opening of the first portion.

14. The thermal support of claim 1, wherein the strap portion is adjustable.

15. The thermal support of claim 1, wherein the thermal insert is reusable.

16. The thermal support of claim 1, wherein the thermal insert forms a floor for the animal to rest on.

17. A method for supporting an animal comprising:
heating the thermal insert of claim 1 to a temperature greater than 30 degrees Celsius or cooling the thermal insert of claim 1 to a temperature less than 10 degrees Celsius;
placing the thermal insert into the pocket of the first portion;
placing the animal into the opening of the first portion; and
placing the strap portion over the shoulder of a user and supporting the animal while the thermal insert transfers heat to or from the animal.

18. The method of claim 17, wherein heating the thermal insert to a temperature greater than 30 degrees Celsius or cooling the thermal insert to a temperature less than 10 degrees Celsius is performed while the thermal insert is outside of the pocket of the first portion.

19. The method of claim 17, wherein the thermal gel portion comprises a thermal gel portion and a support layer, and the method further comprises releasably detaching the thermal gel portion from the support layer prior to heating and/or cooling and/or attaching the thermal gel portion to the support layer prior to placing the thermal insert into the pocket.

20. The method of claim 17, wherein the thermal support is a thermal sling and the first portion is cup shaped or the thermal support is a thermal hoist and the first portion is a U-shaped.
